(19) United States
(12) Patent Application Publication
(10) Pub. No.: US 2022/0346881 A1
(43) Pub. Date: Nov. 3, 2022

(54) SYSTEM AND DEVICE TO PROVIDE UMBILICAL CATHETER TRACKING NAVIGATION AND CONFIRMATION

(71) Applicant: Manikantan Shanmugham, Redmond, WA (US)

(72) Inventor: Manikantan Shanmugham, Redmond, WA (US)

(21) Appl. No.: 17/245,322

(22) Filed: Apr. 30, 2021

(51) Int. Cl. A61B 34/20; A61B 90/00

(52) U.S. Cl. CPC A61B 34/20; A61B 90/37; A61B 2034/2051; A61B 2034/2063

(57) ABSTRACT

An electromagnetic catheter and navigation system may include an umbilical catheter including a first electromagnetic sensor having five degrees of freedom disposed in the tip of the umbilical catheter; an ultrasound imaging system including an ultrasound probe including a second sensor; a non-contact electromagnetic field generator; a sensor interface unit in operable communication with the first electromagnetic sensor; a sensor control unit in operable communication with the sensor interface unit and the non-contact field generator; and a computing device in operable communication with the sensor control unit and the ultrasound imaging system. The system may be constructed and arranged to monitor the location and position of an umbilical catheter after insertion into an umbilical vessel via displaying on a computing device at least one of two-dimensional or three-dimensional imagery indicating the position of the umbilical catheter.

### TECHNICAL FIELD

The embodiments generally relate to the field of umbilical catheters.

### BACKGROUND

An umbilical catheter, such as an umbilical venous catheter (UVC) or an umbilical arterial catheter (UAC), may be used in a newborn or premature child's umbilical cord for monitoring blood pressure, obtaining blood samples, or for emergency vascular access for infusions of fluid or medications. A UAC may be used to assist in breathing, monitoring blood pressure and blood gases, and delivery of medicine. A UVC may be utilized in instances where a newborn is born prematurely, has difficulty feeding, or requires infusion of medicine or an exchange transfusion. The use of an umbilical catheter may allow for repeatedly performing the tasks previously mentioned without repeatedly using a needle on a newborn or prenatal child.

### SUMMARY

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

An electromagnetic catheter and navigation system may provide for a continuous catheter navigation and tracking system constructed and arranged to confirm the location of an umbilical catheter tip after insertion into an umbilical vessel. The system may include a UVC or UAC.

A sensor may be disposed within the tip of the umbilical catheter and may be in operable communication with a sensor interfacing unit via a cable. The system may provide for non-contact magnetic field generation via an electromagnetic field generator positioned above or below a patient. A sensor control unit may be in operable communication with the sensor, the sensor interfacing unit, and a computing device. An ultrasound probe may be used to collect and send images of the patient to a computer to facilitate the tracking of the catheter.

The electromagnetic catheter and navigation system may be constructed and arranged to facilitate the navigation of the tip of the catheter to a target location within a patient without the use of x-rays. The tip of the catheter may be steered by manipulating catheter tip position to facilitate the placement of the catheter in a desired position. The sensor disposed within the tip of the catheter may be a sensor having 5 degrees of freedom (5 DoF) and wherein manipulation may be based on an electromagnetic field signal from the electromagnetic field generator.

An electromagnetic catheter and navigation system may include an umbilical catheter including at least one sensor. The system may further include an ultrasound imaging system, a non-contact electromagnetic field generator, a sensor interface unit in operable communication with the at least one sensor, a sensor control unit in operable communication with the sensor interface unit and the non-contact field generator, and a computing device in operable communication with the sensor control unit and the ultrasound imaging system.

An electromagnetic catheter and navigation system may include an umbilical catheter including a first electromagnetic sensor having five degrees of freedom disposed in the tip of the umbilical catheter. The system may further include an ultrasound imaging system including an ultrasound probe including a second sensor, a non-contact electromagnetic field generator, a sensor interface unit in operable communication with the first electromagnetic sensor, a sensor control unit in operable communication with the sensor interface unit and the non-contact field generator, and a computing device in operable communication with the sensor control unit and the ultrasound imaging system.

A method of monitoring the location and position of an umbilical catheter tip after insertion into an umbilical vessel may include inserting an umbilical catheter into a patient, the umbilical catheter including a first sensor in operable communication with a sensor interface unit in operable communication with a sensor control unit in operable communication with a computing device. The method may further include generating an electromagnetic field near the patient via a non-contact electromagnetic field generator in operable communication with the sensor control unit and monitoring the position of the umbilical catheter within the patient via the sensor control unit and an ultrasound probe in operable communication with an ultrasound unit in operable communication with the computing device.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates an electromagnetic catheter and navigation system according to some variations described herein;

FIG. 2 illustrates an electromagnetic catheter and navigation system according to some variations described herein;

FIG. 3 illustrates an electromagnetic catheter and navigation system according to some variations described herein;

FIG. 4 illustrates an electromagnetic catheter according to some variations described herein;

FIG. 5 illustrates an ultrasound probe according to some variations described herein;

FIG. 6 illustrates a block diagram of an electromagnetic catheter and navigation system according to some variations described herein; and

FIG. 7 illustrates a use case diagram of an electromagnetic catheter and navigation system according to some variations described herein.

### DETAILED DESCRIPTION

The specific details of the single embodiment or variety of embodiments described herein are to the described system and methods of use. Any specific details of the embodiments are used for demonstration purposes only and no unnecessary limitations or inferences are to be understood from there.

It is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

An electromagnetic catheter and navigation system may include a plurality of components including an ultrasound imaging system, at least one sensor disposed within an umbilical catheter, a non-contact field generator, a sensor interface unit, and at least one sensor control unit in operable communication with the sensor interface unit and the non-contact field generator.

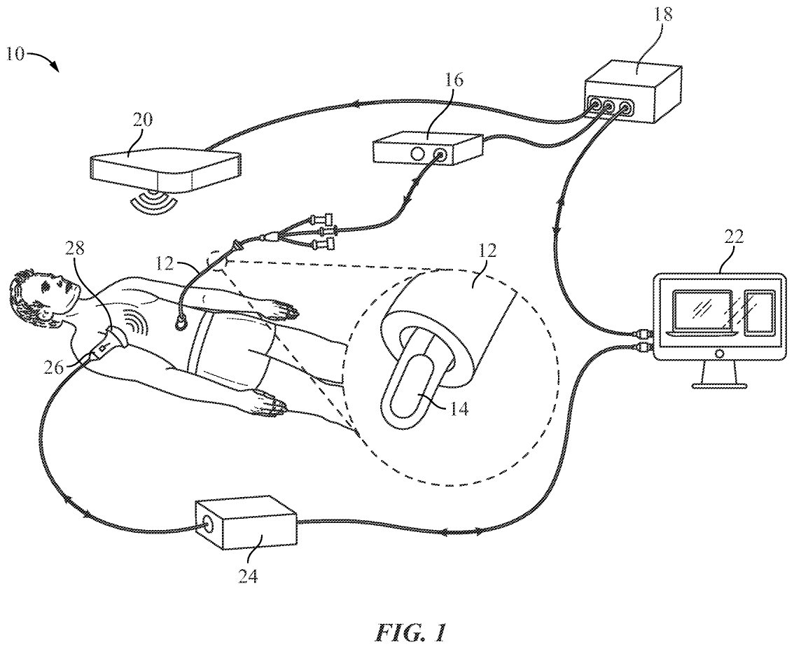

Referring to FIG. 1, an electromagnetic catheter and navigation system 10 may include a catheter 12 including at least one sensor 14 disposed within the tip thereof. The catheter 12 and at least one sensor 14 may be in operable communication with a sensor interface unit 16 in operable communication with a sensor control unit 18. The sensor control unit 18 may be in operable communication with at least one computing device 22 and at least one electromagnetic field generator 20 in addition to the sensor interface unit 16. The at least one computing device 22 may receive information from the sensor control unit 18 and an ultrasound unit 24 in operable communication with an ultrasound probe 26. The ultrasound probe 26 may include a second sensor 28.

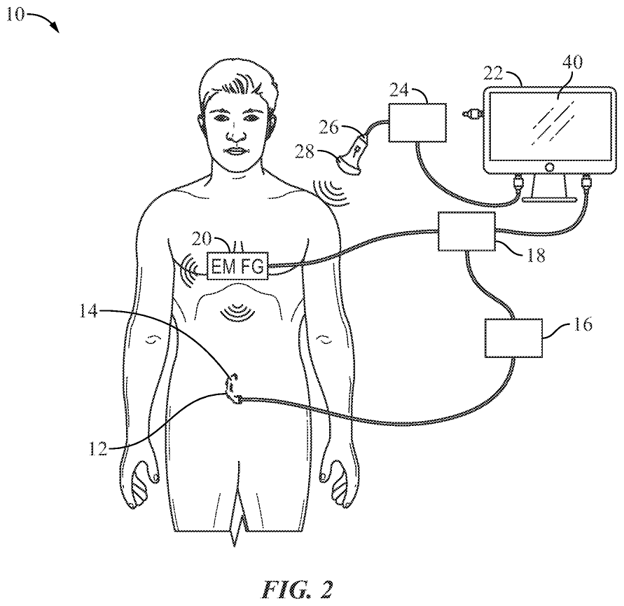

Referring to FIG. 2, an electromagnetic catheter and navigation system 10 may include a catheter 12 including at least one sensor 14 that may have 5 DoF. The catheter 12 and at least one sensor 14 may be in operable communication with a sensor interface unit 16 in operable communication with a sensor control unit 18. The sensor control unit 18 may be in operable communication with at least one computing device 22 and at least one electromagnetic field generator 20. The at least one computing device 22 may receive information from the sensor control unit 18 and an ultrasound unit 24 in operable communication with an ultrasound probe 26. The ultrasound probe 26 may include a second sensor 28. The at least one computing device 22 may receive information from the ultrasound unit 24 and the sensor control unit 18 and display patient information, including catheter positional information, on a display 40.

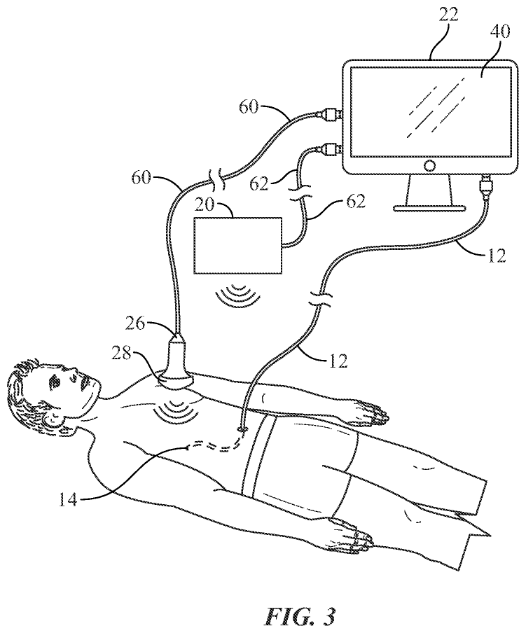

Referring to FIG. 3, a catheter 12 having a sensor 14 disposed within the tip thereof may be tracked within a patient. At least one computing device 22 may receive patient imagery data from an ultrasound probe 26 optionally including a second sensor 28 via an ultrasound connection 60. An electromagnetic field generator 20 may be in operable communication with the computing device 22 via a field generator connection 62 and may facilitate the steering and positioning of the catheter 12 within the body via an electromagnetic signal from the electromagnetic field generator 20. The at least one computing device 22 may receive and display patient information, including catheter positional information, on a display 40.

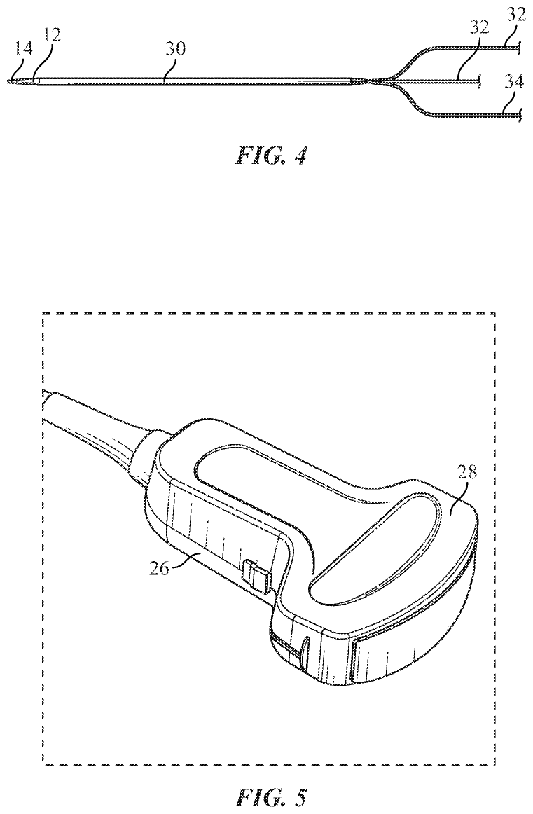

Referring to FIG. 4, an electromagnetic catheter 12 may include electrical leads 32 and a monofilament 34 encapsulated in heat shrink tubing 30. The monofilament 34 may be attached to the tip of a 5 DoF sensor 14 to facilitate integration with the catheter 12 and may function as a pull wire and cut off after the 5 DoF sensor 14 has been positioned within the body.

Referring to FIG. 5, an ultrasound probe 26 may include a second sensor 28 having 5 DoF corresponding to the ultrasound probe 26 and ultrasound unit 26 as depicted in FIG. 1-3. The second sensor 28 may further facilitate the tracking of a first sensor within a catheter (e.g., the sensor 14 depicted within the catheter 12 of FIG. 4) as displayed on the display 40.

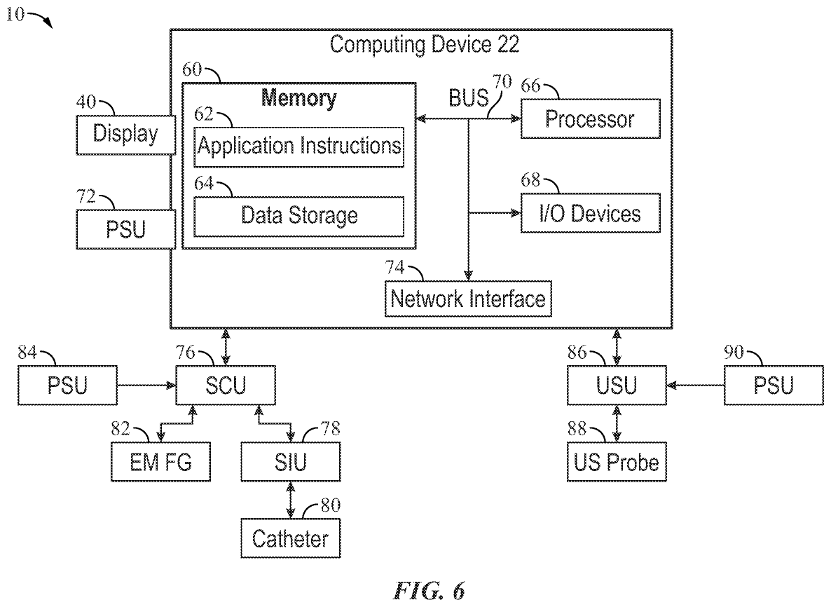

Referring to FIG. 6, an electromagnetic catheter and navigation system 10 may include a computing device 22. The computing device 22 may include memory 60, at least one processor 66, various input and output (I/O) devices 68 and may optionally be interfaced with a network via a network interface 74. The memory 60 may include data storage 64 and, optionally, application instructions 62. The application instructions 62 may be constructed and arranged to receive ultrasound data, electromagnetic field generator data, and with various components of the system to facilitate the display of said data including two-dimensional or three-dimensional visuals of a patient's body and sensor position information. The computing device 22 may include one or more processors 66 in operable communication with the memory 60 through a system bus 70. The bus 70 may additionally couple various system components. The bus 70 may be any of several types of bus structures including a memory bus or memory controller, a peripheral bus, and a local bus using any of a variety of bus architectures. The computing device 22 may include a display 40 and a power supply unit (PSU) 72 constructed and arranged to power the computing device 22. The computing device 22 may be in operable communication with a sensor control unit (SCU) 76, which may optionally include its own power supply unit 84. An electromagnetic field generator (EM FG) 82 may be in operable communication with the sensor control unit 76 such that the computing device 22 may facilitate the control of the electromagnetic field generator 82. A sensor interface unit (SIU) 78 in operable communication with a catheter 80 that may also be in operable communication with the sensor control unit 76 such that the computing device 22 may be used to monitor the position of the catheter 80. An ultrasound unit (USU) 86 may optionally include a power supply unit 90 and may include an ultrasound probe 88 that may include a second sensor as in FIG. 5. The ultrasound unit 86 may be in operable communication with the computing device 22 such that the computing device 22 may receive ultrasound data via the ultrasound probe 88.

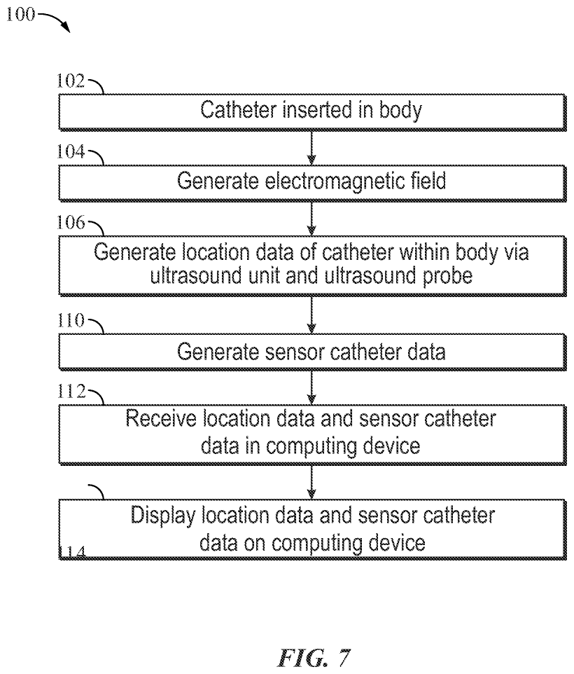

Referring to FIG. 7, a method 100 of using an electromagnetic catheter and the navigation system may include inserting or providing a catheter in a patient's body 102. The method may include generating or providing an electromagnetic field via a non-contact electromagnetic field generator 104. The method may include collecting location data of the catheter within the patient's body via an ultrasound unit and ultrasound probe 106 in operable communication with a computing device. The method may include generating sensor data 110. The method may include receiving location data, and sensor data in a computing device 112 and displaying location data, and sensor data on a display of the computing device 114.

The electromagnetic catheter and the navigation system may provide for a continuous catheter navigation and tracking system constructed and arranged to monitor and control the location and position of an umbilical catheter tip after insertion into an umbilical vessel. A first sensor having 5 DoF may be disposed within the tip of the umbilical catheter and may be in operable communication with a sensor interfacing unit via a cable. The system may provide for non-contact magnetic field generation via an electromagnetic field generator positioned above, below, or adjacent a patient. A sensor control unit may be in operable communication with the first sensor, the sensor interfacing unit, and a computing device. The tip of the catheter may be steered and positioned within the body via an electromagnetic signal from a non-contact electromagnetic field generator by manipulating the catheter tip position to facilitate the placement of the catheter in a desired position. An ultrasound probe may be used to collect and send images of the patient and catheter to the computer to facilitate the tracking of the catheter. The sensor control unit and ultrasound unit may be constructed and arranged to provide two-dimensional imagery or three-dimensional imagery of the position of the catheter and display such on a display of a computing device. The ultrasound probe may include a second sensor having 5 DoF to further facilitate the tracking of the first sensor.

The ultrasound imaging system may be based on the reflection of ultrasound waves within the body to produce internal imagery of the body. The ultrasound imaging system may include an ultrasound unit in operable communication with an ultrasounds probe and may be in operable communication with a computing device such as a computer or smart device. An ultrasound probe head may emit ultrasound waves into a patient's body and reflections of the ultrasound waves from specific types of tissue having different acoustic impedance levels may return to the ultrasound probe head to provide internal imagery of the body. The ultrasound imaging system in combination with the ultrasound probe head may be used to track the location of the umbilical catheter within the body.

At least one sensor may be disposed within an umbilical catheter. The at least one sensor may be an electromagnetic sensor having 5 DoF. The at least one sensor may be a coil-type sensor. A 5 DoF sensor may be constructed and arranged to measure translation in three degrees of freedom and two rotational directions relative to the longitudinal axis of the sensor. That is, the least one sensor may provide orientation and position data with respect to x, y, and z position in addition to pitch and yaw data. The at least one sensor may include electrical leads encapsulated in heat shrink tubing. A mono filament may be attached to the sensor's tip to facilitate integration with the catheter and may function as a pull wire and cut off after the at least one sensor has been positioned within the body. Additionally, at least one second sensor may be disposed within the ultrasound probe and may be constructed and arranged to provide relative positional data to a user with respect to the depth of the catheter within the human body relative to the ultrasound probe.

A non-contact electromagnetic field generator may be constructed and arranged for spatial measurement of the at least one sensor to provide accurate tracking of 5 DoF. The non-contact magnetic field generator may produce varying magnetic fields to create a defined volume of varying magnetic flux in which sensors can be tracked with a high degree of accuracy. The non-contact electromagnetic field generator may be positioned above, below, or generally nearby a patient's body to assist in the tracking of the at least one sensor. The non-contact electromagnetic field generator may be in operable communication with the sensor control unit.

A sensor control unit may facilitate the operation the electromagnetic catheter and navigation system. The at least one sensor control unit may be in operable communication with at least one computing device constructed and arranged to receive information from the sensor control unit. The sensor control unit may provide power to the sensor interface unit and the non-contact field generator, may receive sensor data via the sensor interface unit and may determine sensor position and orientation. The sensor control unit may report positional and orientation data of the at least one sensor to a computer. The sensor control unit may be in operable communication with the sensor interface unit, the non-contact field generator, and at least one computing device.

A sensor interface unit may be constructed and arranged to interface between the at least one sensor, the second sensor, and the system control unit. The sensor interface unit may be constructed and arranged to convert analog signals produced by the at least one sensor and second sensor to digital signals which may be received by the sensor control unit.

It is contemplated that the arrangement of the ultrasound imaging system, at least one sensor disposed within an umbilical catheter, a non-contact field generator, a sensor interface unit, and at least one sensor control unit may include a variety of different combinations and the examples listed herein shall not be considered limiting.

The following description of variants is only illustrative of components, elements, acts, products, and methods considered to be within the scope of the invention and are not in any way intended to limit such scope by what is specifically disclosed or not expressly set forth. The components, elements, acts, products, and methods as described herein may be combined and rearranged other than as expressly described herein and are still considered to be within the scope of the invention.

According to variation 1, an electromagnetic catheter and navigation system may include an umbilical catheter including at least one sensor; an ultrasound imaging system; a non-contact electromagnetic field generator; a sensor interface unit in operable communication with the at least one sensor; a sensor control unit in operable communication with the sensor interface unit and the non-contact field generator; and a computing device in operable communication with the sensor control unit and the ultrasound imaging system.

Variation 2 may include an electromagnetic catheter and navigation system as in variation 1, wherein the at least one sensor is disposed within a tip of the umbilical catheter.

Variation 3 may include an electromagnetic catheter and navigation system as in any of variations 1 through 2, wherein the at least one sensor is an electromagnetic sensor having five degrees of freedom.

Variation 4 may include an electromagnetic catheter and navigation system as in any of variations 1 through 3, wherein the at least one sensor is a coil-type sensor.

Variation 5 may include an electromagnetic catheter and navigation system as in any of variations 1 through 4, wherein the ultrasound imaging system further includes an ultrasound probe.

Variation 6 may include an electromagnetic catheter and navigation system as in any of variations 1 through 5, wherein the ultrasound probe includes a second sensor.

Variation 7 may include an electromagnetic catheter and navigation system as in any of variations 1 through 6, wherein the electromagnetic catheter and navigation system is constructed and arranged to monitor the location and position of an umbilical catheter after insertion into an umbilical vessel.

Variation 8 may include an electromagnetic catheter and navigation system as in any of variations 1 through 7, wherein the electromagnetic catheter and navigation system is constructed and arranged to monitor the location and position of an umbilical catheter after insertion into an umbilical vessel via displaying on the computing device two-dimensional imagery indicating the position of the umbilical catheter.

Variation 9 may include an electromagnetic catheter and navigation system as in any of variations 1 through 8, wherein the electromagnetic catheter and navigation system is constructed and arranged to monitor the location and position of an umbilical catheter after insertion into an umbilical vessel via displaying on the computing device three-dimensional imagery indicating the position of the umbilical catheter.

According to variation 10, an electromagnetic catheter and navigation system may include an umbilical catheter including a first electromagnetic sensor having five degrees of freedom disposed in an umbilical catheter; an ultrasound imaging system including an ultrasound probe including a second sensor; a non-contact electromagnetic field generator; a sensor interface unit in operable communication with the first electromagnetic sensor; a sensor control unit in operable communication with the sensor interface unit and the non-contact field generator; and a computing device in operable communication with the sensor control unit and the ultrasound imaging system.

Variation 11 may include an electromagnetic catheter and navigation system as in variation 10, wherein the electromagnetic catheter and navigation system is constructed and arranged to monitor the location and position of the umbilical catheter after insertion into an umbilical vessel via displaying on the computing device at least one of two-dimensional or three-dimensional imagery indicating the position of the umbilical catheter.

Variation 12 may include an electromagnetic catheter and navigation system as in any of variations 10 through 11, wherein the first electromagnetic sensor is a coil-type sensor.

According to variation 13, a method of monitoring the location and position of an umbilical catheter after insertion into an umbilical vessel, including inserting an umbilical catheter into a patient, the umbilical catheter including a first sensor in operable communication with a sensor interface unit in operable communication with a sensor control unit in operable communication with a computing device; generating an electromagnetic field near the patient via a non-contact electromagnetic field generator in operable communication with the sensor control unit; and monitoring the position of the umbilical catheter within the patient via the sensor control unit and an ultrasound probe in operable communication with an ultrasound unit in operable communication with the computing device.

Variation 14 may include a method of monitoring the location and position of an umbilical catheter after insertion into an umbilical vessel as in variation 13, further including displaying on the computing device two-dimensional imagery indicating the position of the umbilical catheter.

Variation 15 may include a method of monitoring the location and position of an umbilical catheter after insertion into an umbilical vessel as in any of variations 13 through 14, further including displaying on the computing device three-dimensional imagery indicating the position of the umbilical catheter.

Variation 16 may include a method of monitoring the location and position of an umbilical catheter after insertion into an umbilical vessel as in any of variations 13 through 15, wherein the at least one sensor is disposed within a tip of the umbilical catheter.

Variation 17 may include a method of monitoring the location and position of an umbilical catheter after insertion into an umbilical vessel as in any of variations 13 through 16, wherein the at least one sensor is an electromagnetic sensor having five degrees of freedom.

Variation 18 may include a method of monitoring the location and position of an umbilical catheter after insertion into an umbilical vessel as in any of variations 13 through 17, wherein the at least one sensor is a coil-type sensor.

Variation 19 may include a method of monitoring the location and position of an umbilical catheter after insertion into an umbilical vessel as in any of variations 13 through 18, wherein the ultrasound probe includes a second sensor.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for anyone of the elements in the claims below or that a single element can be substituted for two or more elements in a claim. Although elements can be described above as acting in certain combinations, and even initially claimed as such, it is to be expressly understood that one or more elements from a claimed combination can, in some cases, be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible considering the above teachings without departing from the following claims.

What is claimed is:

1. An electromagnetic catheter and navigation system comprising:
an umbilical catheter comprising at least one sensor;
an ultrasound imaging system;
a non-contact electromagnetic field generator;
a sensor interface unit in operable communication with the at least one sensor;
a sensor control unit in operable communication with the sensor interface unit and the non-contact electromagnetic field generator; and
a computing device in operable communication with the sensor control unit and the ultrasound imaging system.

2. An electromagnetic catheter and navigation system as set forth in claim 1, wherein the at least one sensor is disposed within a tip of the umbilical catheter.

3. An electromagnetic catheter and navigation system as set forth in claim 1, wherein the at least one sensor is an electromagnetic sensor having five degrees of freedom.

4. An electromagnetic catheter and navigation system as set forth in claim 1, wherein the at least one sensor is a coil-type sensor.

5. An electromagnetic catheter and navigation system as set forth in claim 1, wherein the ultrasound imaging system further comprises an ultrasound probe.

6. An electromagnetic catheter and navigation system as set forth in claim 5, wherein the ultrasound probe comprises a second sensor.

7. An electromagnetic catheter and navigation system as set forth in claim 1, wherein the electromagnetic catheter and navigation system is constructed and arranged to monitor the location and position of an umbilical catheter after insertion into an umbilical vessel.

8. An electromagnetic catheter and navigation system as set forth in claim 1, wherein the electromagnetic catheter and navigation system is constructed and arranged to monitor the location and position of an umbilical catheter after insertion into an umbilical vessel via displaying on the computing device two-dimensional imagery indicating the position of the umbilical catheter.

9. An electromagnetic catheter and navigation system as set forth in claim 1, wherein the electromagnetic catheter and navigation system is constructed and arranged to monitor the location and position of an umbilical catheter after insertion into an umbilical vessel via displaying on the computing device three-dimensional imagery indicating the position of the umbilical catheter.

10. An electromagnetic catheter and navigation system comprising:
an umbilical catheter comprising a first electromagnetic sensor having five degrees of freedom disposed in an umbilical catheter;
an ultrasound imaging system comprising an ultrasound probe comprising a second sensor;
a non-contact electromagnetic field generator;
a sensor interface unit in operable communication with the first electromagnetic sensor;
a sensor control unit in operable communication with the sensor interface unit and the non-contact electromagnetic field generator; and
a computing device in operable communication with the sensor control unit and the ultrasound imaging system.

11. An electromagnetic catheter and navigation system as set forth in claim 10, wherein the electromagnetic catheter and navigation system is constructed and arranged to monitor the location and position of the umbilical catheter after insertion into an umbilical vessel via displaying on the computing device at least one of two-dimensional or three-dimensional imagery indicating the position of the umbilical catheter.

12. An electromagnetic catheter and navigation system as set forth in claim 10, wherein the first electromagnetic sensor is a coil-type sensor.

13. A method of monitoring the location and position of an umbilical catheter after insertion into an umbilical vessel, comprising:
inserting an umbilical catheter into a patient, the umbilical catheter comprising a first sensor in operable communication with a sensor interface unit in operable communication with a sensor control unit in operable communication with a computing device;
generating an electromagnetic field near the patient via a non-contact electromagnetic field generator in operable communication with the sensor control unit; and
monitoring the position of the umbilical catheter within the patient via the sensor control unit and an ultrasound probe in operable communication with an ultrasound unit in operable communication with the computing device.

14. A method of monitoring the location and position of an umbilical catheter after insertion into an umbilical vessel as set forth in claim 13, further comprising displaying on the computing device two-dimensional imagery indicating the position of the umbilical catheter.

15. A method of monitoring the location and position of an umbilical catheter after insertion into an umbilical vessel as set forth in claim 13, further comprising displaying on the computing device three-dimensional imagery indicating the position of the umbilical catheter.

16. A method of monitoring the location and position of an umbilical catheter after insertion into an umbilical vessel as set forth in claim 13, wherein the at least one sensor is disposed within the umbilical catheter.

17. A method of monitoring the location and position of an umbilical catheter after insertion into an umbilical vessel as set forth in claim 13, wherein the at least one sensor is an electromagnetic sensor having five degrees of freedom.

18. A method of monitoring the location and position of an umbilical catheter after insertion into an umbilical vessel as set forth in claim 13, wherein the at least one sensor is a coil-type sensor.

19. A method of monitoring the location and position of an umbilical catheter after insertion into an umbilical vessel as set forth in claim 13, wherein the ultrasound probe comprises a second sensor.
